(19) United States
(12) Patent Application Publication
(10) Pub. No.: US 2021/0388303 A1
(43) Pub. Date: Dec. 16, 2021

(54) Bioreactor with Filter

(71) Applicant: Global Life Sciences Solutions USA LLC, Marlborough, MA (US)

(72) Inventors: Mathew Wecharatana, Marlboro, MA (US); Yasser Ali, Marlborough, MA (US); Donald Fraser, Marlborough, MA (US); James Brueggeman, Marlborough, MA (US)

(21) Appl. No.: 17/292,288

(22) PCT Filed: Nov. 27, 2019
(86) PCT No.: PCT/EP2019/082722

§ 371 Date: May 7, 2021

Related U.S. Application Data
(60) Provisional application No. 62/772,175, filed on Nov. 28, 2018.

(51) Int. Cl. C12M 1/00; C12M 3/06; C12M 1/26

(52) U.S. Cl. CPC C12M 29/10; C12M 23/26; C12M 23/14; C12M 33/14; C12M 27/16

(57) ABSTRACT

A cellbag bioreactor includes a stacked filter providing multiple porous membranes to define a filter cavity. Additionally, a filter within the cellbag bioreactor may be tethered so as to help maintain each membrane of a filter wetted during bioreactor operations.

### FIELD OF THE INVENTION

The present invention relates to the field of bioprocessing technology. More specifically, the present invention relates to bioreactors with one or more filters disposed therein.

### BACKGROUND OF THE INVENTION

Culturing of cells for producing regenerative medicine is done with the aim of harvesting cells which can subsequently be injected into a patient. The health and viability of the cells is of paramount importance. The cells need to be reproduced under controlled conditions and fed with nutrients to grow. One commercially successful disposable bioreactor system uses a flexible cellbag bioreactor placed on a rockable platform. The bioreactor is partially filled with liquid cell culture medium and cells of interest are introduced into the bioreactor. The culture medium and cells contact only a presterile, disposable chamber that is positioned on the rocking platform. The rocking motion of the platform induces waves in the culture fluid and thereby provides continual mixing and oxygen transfer, resulting in a robust environment for cell growth. The Bioreactor requires no cleaning or sterilization and provides ease of operation and protection against cross-contamination.

A perfusion bioreactor grows cells by continuously feeding the bioreactor with fresh cell culture medium so as to replace the spent cell culture medium all while keeping the volume of the cell culture medium constant in the bioreactor. The cells reach a steady state of reproduction and can be maintained in that state for a few weeks until the required cell density is achieved. Perfusion bioreactors typically employ a filter within the bioreactor for filtering out of the bioreactor both the spent cell culture medium and the toxic cell metabolites that inhibit cell growth while retaining the healthy and viable cells within the bioreactor.

One type of perfusion bioreactor is disclosed in U.S. Pat. No. 9,017,997B2, where the filter is affixed to the inner surface of a bottom wall of the bioreactor and thus does not float on the surface of the culture medium. This placement of the filter prevents the filter from getting damaged due to twisting or sticking to the walls of the bioreactor. However, the filter can get easily clogged and fouled. Similarly, WO2012/158108A1 discloses a perfusion bioreactor for cultivation of cells on microcarriers where the filter is fixed to the inner surface of a wall of the bioreactor. Further, WO2015/034416A1 discloses a bioreactor with internal dialysis modules suitable for dialysis cultivation of cells. The dialysis compartments are formed as either a freely movable bundle of hollow fiber membranes, a pouch attached to an inner wall of the bag, a pouch freely moving, or a sheet of membrane fixed to an inner wall of the bag.

Another perfusion bioreactor is disclosed in WO2017/055059A1, where the filter is held by a filter holding device. The filter holding device is attached to an inner wall of the bioreactor such that there is a limited space between the filter and the inner wall of the bioreactor and the liquid medium provided in the bioreactor can flow on both sides of the filter. While this arrangement of the filter holding device causes a cross flow filtration effect, it is quite limited in reducing the clogging and fouling of the filter due to limited space between the filter and the inner wall of the bioreactor.

In the perfusion bioreactor disclosed in U.S. Pat. No. 6,544,788, the perfusion filter is constructed to move freely on top of the liquid cell culture medium during the rocking motion of the bioreactor. FIG. 1 depicts a typical filter 10 used in such prior art. Filter 10 employs a stacked material design where a porous planar membrane 12 forms the bottom surface of the stack, a relatively rigid planar mesh 14 forms the middle layer, and a fluid-impervious planar film 16 forms the top layer. Mesh 14 extends within a filter cavity 15 defined between the opposing surfaces of membrane 12 and film 16. Film 16 is used to back porous membrane 12 and includes an evacuation port 18 as an avenue for waste material filtered up through membrane 12 into the filter cavity 15 to be directed out filter 10 via an elongate hollow conduit 20 extending from port 18 to a port formed on a top surface of the bioreactor. Filter 10 is tethered to the top surface of the bioreactor by evacuation conduit 20. The rocking motion of the bioreactor keeps the filter from clogging due to the erosion of any debris by the turbulence generated by the tangential motion of the filter relative to the cell culture medium. However, as the filter is constructed to move freely, it can lead to twisting and turning of the filter thereby damaging it. This design can also cause the filter to stick to the inner walls of the bioreactor thus impairing the gaseous exchange and filtration of the cell culture medium. Also, when the filter is floating on the surface of the cell culture medium, the prefusion process may fail if the entire membrane surface is not wetted out. For example, if the filter membrane bows such that the entire membrane is not fully exposed to the cell culture medium. The evacuation pump will then also draw air instead of just the cell culture medium which can lead to a progressive inflation of the waste collection bag with air instead of spent cell culture medium as well as variable perfusion rates. This phenomenon of pulling air through the filter is called ‘bubbling’. Mitigation of bubbling can require either manually pressing on the filter through the bioreactor bag when it is partially-full of fluid so as to fully submerge the filter or to alter the process so as to completely fill the bioreactor bag with fluid so as to submerge and fully wet the filter prior to withdrawing fluid before starting the bioreactor process.

### SUMMARY OF THE INVENTION

The present invention provides a stacked filter design which incorporates a porous membrane at both major surfaces of the filter stack rather than only a single side. The design allows for reduction of the overall footprint of the filter, as waste can be pumped through from both top and bottom membrane surfaces, i.e., waste can be drawn from both above and below the filter. In addition, by maintaining both membranes in a wetted state, the filter design mitigates the potential for air to be pumped through, as any wetted area would pull fluid into the filter stack and then out of the cellbag bioreactor.

During a cell expansion operation in which cells are perfused, the filter of the present invention can allow more fluid exposure to the membrane surface and therefore increased volume of waste to be admitted into the filter pumped from the bioreactor. Additionally, optimization of the filter size can also reduce the overall footprint of the filter in the bioreactor. Moreover, by placing a port on the bottom of the filter stack rather than the top, the filter design reduces the potential for air to be pumped through, as any wetted area would pull fluid into the filter stack and to the bottom side port.

The present invention further provides a perfusion filter tethered from the bottom surface of the cellbag bioreactor, thus providing an anchor point for the filter that is within the fluid volume. Controlling the length of the tether will allow the filter to remain wetted throughout the culture process, which mitigates the risk of the filter floating to the surface and parts of the filter not being exposed to fluid or otherwise wetted out.

Alternatively, the present invention can tether the filter to the bottom of the bioreactor bag by selectively tethering the waste conduit to the bottom surface of the bioreactor bag, allowing a portion of the conduit proximate the filter to rise up from the bottom surface so as to maintain the filter within a predefined constrained volume of the bioreactor chamber such that the membranes of the filter remain wetted.

Desirably, the filter and tethers of the present invention will be used in cell therapy and bioprocessing applications so as to minimize the risk of a filter floating on the top surface of a culture volume and sucking in air. The present invention reduces risk and the need for manual manipulation of the bioreactor throughout the process. Moreover, as the present invention obviates the need to manually manipulate the bag to ensure wetting, or to change unit operations requiring the bioreactor be filled with liquid before inflating, user processes can be streamlined.

### BRIEF DESCRIPTION OF THE DRAWINGS

FIG. 1 shows a cross-sectional view of a bioreactor filter of the prior art.

FIG. 2 shows a bottom-oblique view of the filter of the present invention.

FIG. 3 shows a cross-sectional view of the bioreactor filter of FIG. 2 taken through the line X-X.

FIG. 4 shows a bottom exploded view of a filter of FIG. 2.

FIG. 5 shows a top-oblique-view of a cellbag bioreactor with a filter of the present invention.

FIG. 6 depicts a cellbag bioreactor of the present invention on a rocking platform as part of a bioreactor system.

FIG. 7 shows a side view of a cellbag bioreactor of the present invention employing tethers to a filter of the present invention.

FIG. 8 shows a cellbag bioreactor of the present invention which tethers the fluid conduit below the bioreactor filter

FIG. 9 shows a cellbag bioreactor which employs an alternate tether of the present invention.

FIG. 10 depicts a close-up view of the tether of FIG. 9.

FIG. 11 depicts another cellbag bioreactor of the present invention which tethers a filter so as to pull waste fluid below the filter prior to exiting an upper surface of the bioreactor.

FIG. 12 depicts a bottom oblique view of an alternate filter of the present invention.

FIG. 13 depicts a partial oblique exploded view of the filter of FIG. 12.

FIG. 14 depicts a cross-sectional view of the filter of FIG. 12 taken through the line Y-Y.

FIG. 15 depicts a bioreactor incorporating a filter of FIG. 12.

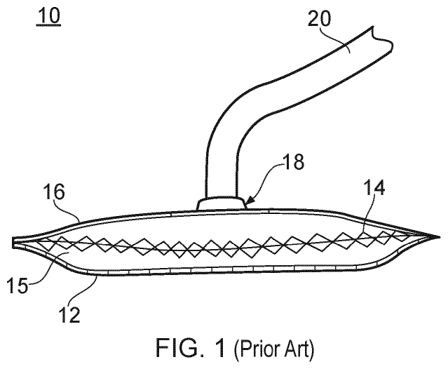

### DETAILED DESCRIPTION OF THE PREFERRED EMBODIMENTS

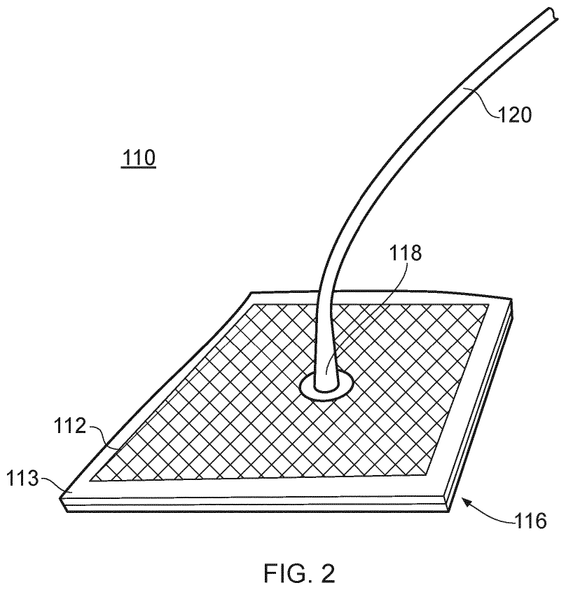

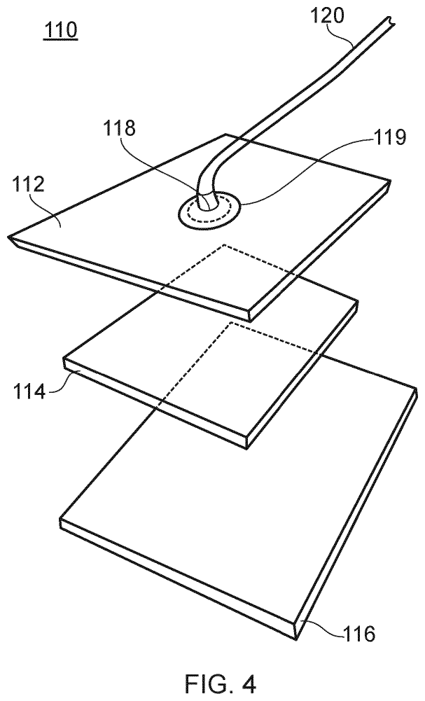

FIGS. 2-4 depict an exemplary stacked filter 110 of the present invention. Filter 110 includes first and second planar porous membranes 112 and 116 perimetrically bonded, along edge 113 about a substantially planar open mesh 114 which extends across a filter cavity 115 defined between unbonded portions of membranes 112 and 116. Filter 110 further comprises a port 118 on bottom membrane 112. Desirably, port 118 is an open fitment attached to membrane 112 about an enlarged aperture 119 (shown in phantom lines in FIG. 4) defined by membrane 112. Port 118 is connected to an elongate hollow fluid conduit 120 that extends to a second port located on a surface of a bioreactor. Conduit 120 is thus able to conduct waste which has transited membranes 112 and 116 into cavity 115 out through port 118 and thus out of its respective bioreactor. Port 118 typically include a short hollow cylindrical section with an annular rim projecting out from one end there, as is known in the art. The annular rim may be bonded to either major surface of membrane 112 such that port 118 may be said to either be bonded onto membrane 112 or to extend through membrane 112 at aperture 119 to make fluid-tight connection to conduit 120.

Each of membrane 112 and 116, mesh 114, port 118, and conduit 120 are formed of a biologically-compatible hydrophilic material suitable for bioprocessing operations as are known in the art. The perimetrical bonding of membranes 112 and 116, as well as the bonding of port 118 and conduit 120 are by means suitable for bioprocessing operations and compatible for pharmaceutical operations, as is known in the art. By way of illustration and not of limitation, membranes 112 and 116 may be formed of Ultra-high-molecular-weight polyethylene (UHMWPE), Nylon or Polyethersulfone (PE), mesh 114 may be formed of Polyethylene terephthalate (PETE), while port 118 and conduit 120 may be formed of any suitable plastic or rubber material. Filter 110 may be used in cell therapy and bioprocessing operations to reduce the risk for air to be pumped to waste during perfusion processes rather than waste material. The perfusion filter design of the present invention, with both top and bottom membranes, provides more surface areas for cells to migrate through to waste and when used in conjunction with other features, can resolve any air being pumped into the waste bag. Openings 114a formed in mesh 114 are larger than the pores 112a and 116a of membranes 112 and 116, respectively. It is contemplated that mesh 114 may approximate an interwoven lattice structure or any other structure which provides separation between membranes 112 and 116 while also allowing for the permeate which has passed through either membrane 112 or 116 to traverse filter cavity 115 to be conducted through aperture 119 and out conduit 120.

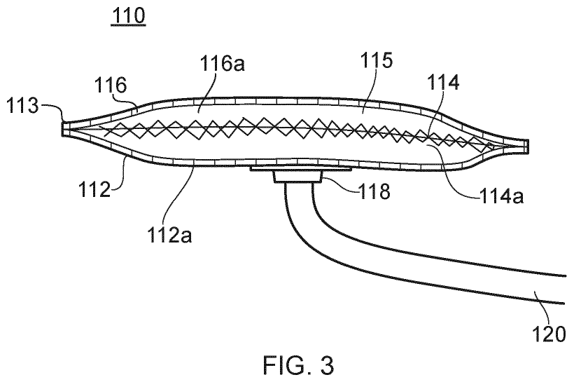

FIGS. 2 and 4 depict filter 110 from a bottom perspective while FIG. 3 depicts filter 110 in a relatively inverted orientation. As will be shown hereinbelow, the present invention contemplates that filter 110 may be employed in either orientation, i.e., with port 118 and conduit 120 protruding and extending either above or below filter 110 (where the terms ‘above’, ‘upper’, and ‘top’ and ‘below’, ‘lower’, and ‘bottom’ are ascribed throughout with respect to a corresponding gravitational vector generally extending from the top of the page—i.e., corresponding to ‘above’, ‘upper’, and ‘top’—towards the bottom of the page—i.e., corresponding to ‘below’, ‘lower’, and ‘bottom’). For purposes of this description, regardless of the orientation of filter 110 in operation, membrane 112 will always support port 118 while membrane 116 will always be located opposite filter cavity 115 from port 118.

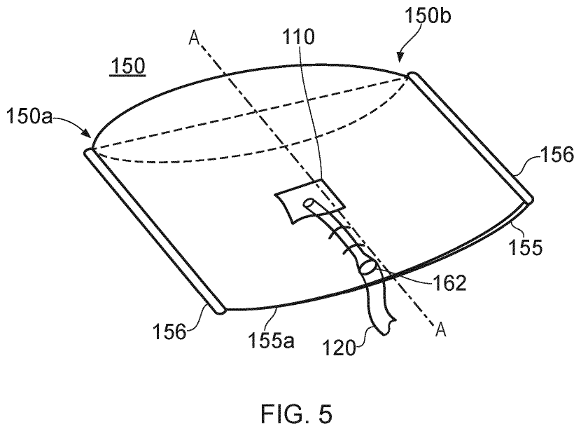

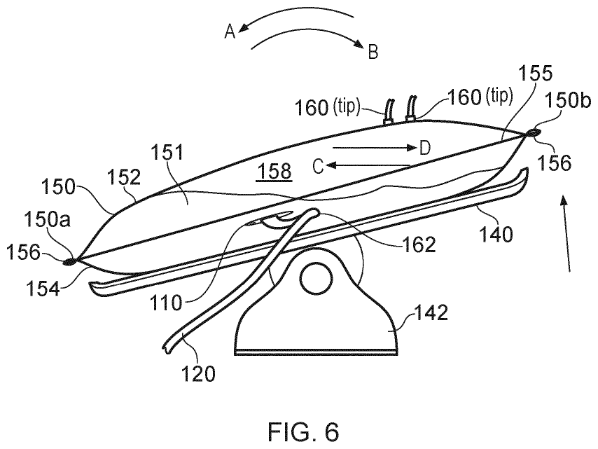

FIG. 5 depicts a cellbag bioreactor (also described as a bioreactor) 150 according to a first embodiment of the invention. Bioreactor 150 is desirably one component of a single-use bioreactor system, such is used with the WAVE BIOREACTOR® sold by GE Healthcare Life Sciences. With additional reference to FIG. 6, the bioreactor system includes a rocker platform 140 pivotally connected to a base 142, the cellbag bioreactor 150, and an aeration/inflation pump and various nutrition and sensor connections (not shown). Bioreactor 150 is made of a flexible material and comprises a substantially planar top layer 152, a substantially planar bottom layer 154 and a filter 110. Layers 152 and 154 are perimetrically-bonded along perimetrical edge 155 so as to define an inflatable bioreactor chamber 158. For stability, layers 152 and 154 may also enclose a pair of substantially rigid elongate support rods 156 at opposed longitudinal ends 150a and 150b, respectively, of bioreactor 150.

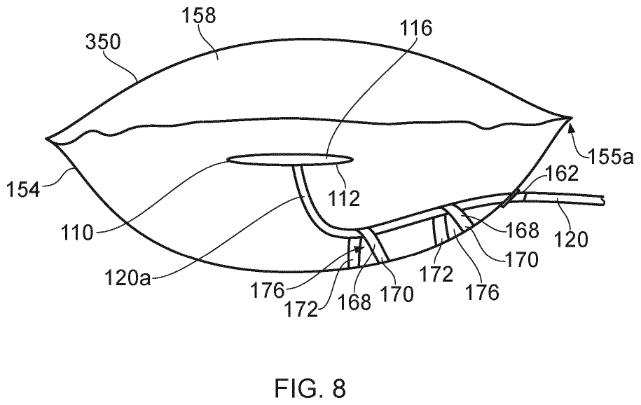

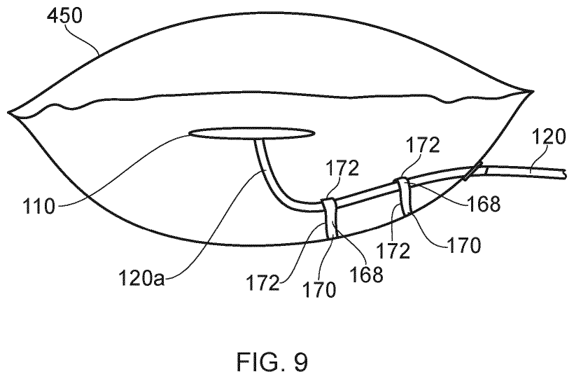

Filter 110 is located within chamber 158 so that waster material may be conducted through conduit 120 out of bioreactor 150. Platform 140 is urged to rock back and forth in the directions of Arrows A and B so as to impart counter-flowing waves of the cell media 151 within bioreactor 150 in the respective directions of Arrows C and D. The necessary oxygen and nutrients are provided to for cell growth and productivity. Top layer 152 and bottom layer 154 of the bioreactor 150 are formed from a suitable material for bioprocessing such as, by way of illustration but not of limitation, a multilayer laminated clear film of EVA. Top layer 152 and bottom layer 154 are further typically formed from a transparent or semitransparent multilayer laminated film which allows for an operator to generally observe bioreactor chamber 158. Top layer 152 supports multiple access ports 160 which when properly connected provide access to the bioreactor chamber 158 for needed nutrients, oxygen, or sensors. For example, one port 160 could be used to transfer fresh liquid media from the external space of the bioreactor 150 to the bioreactor chamber 158 while another port 160 could be connected to an oxygen level sensor. Bioreactor 150 further supports a perfusion port 162 through which conduit 120 transits top from chamber 158. The present invention contemplates that both membranes 112 and 116 of filter 110 are properly wetted before drawing waste from media 164 through filter 110 into conduit 120. With additional reference to FIGS. 8 and 9, although bioreactor port 162 may be positioned on bottom layer 154, given the curvature of the bag when inflated, this port is desirably placed in a location close to the lateral centerline A-A of bioreactor 150 and close to a lateral edge segment 155a so that platform 140 does not interfere with flow through conduit 120 during operation.

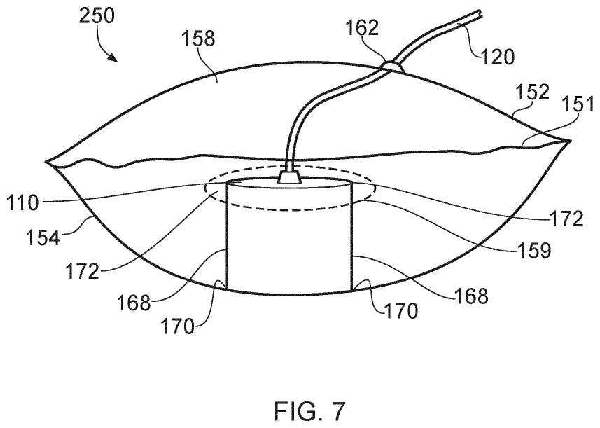

Referring to FIG. 7, the present invention further contemplates a bioreactor 250 including one or more elongate tethers 168 having opposed first and second ends 170 and 172, respectively, and an elongate tether body 174 extending therebetween. Bioreactor 250 is contemplated to be similar to bioreactor 150, with like references indicating like components and with modifications as noted. First ends 170 of tethers 168 are desirably bonded to bottom layer 154 while second ends 172 are bonded to perimetrical edge 113 of filter 110. The lengths of tethers 168 are selected so as to maintain filter 110 within a set distance from bottom layer 154. Desirably, tethers 168 are able to maintain both membranes 112 and 116 of filter 110 wetted during evacuation of waste from bioreactor 110 to prevent bubbling. Tethers 168 should also provide a slender profile about which the media flows during rocking of bioreactor 150 so as to not create zones of immobile eddy's which prevent entrapped media from flowing past the tethers. Each tether 168 of the present invention is desirably formed of a flexible strip of a polymer suitable for use in bioprocessing including, by way of illustration and not of limitation, EVA. Desirably, tethers 168 provide limited movement of filter 110 within a predefined constrained volume 159 (depicted by dashed-lines) of chamber 158 when bioreactor 150 is filled at least partially with a liquid media as is known for bioprocessing operations so as to maintain the wetting of both membranes 112 and 116.

Tethers 168 constrain the movement of filter 110 but allow some movement of filter 110 vertically, i.e., towards and away from bottom layer 154, laterally, towards and away from edge portion 155a, and longitudinally towards and away from longitudinal ends 150a and 150b such that the movement of the filter is within a constrained volume 159 of the overall chamber 158 which can be predefined by the amount of slack, flexibility or elasticity of each tether. Ideally constrained volume 159 is spaced from the inner surface of a cell bag in use to avoid the filter rubbing on the cell bag in use. Constrained volume 159 will thus change in use as the amount and volume of liquid in chamber 158 changes and in accordance with the length, positioning, and flexibility of the tethers 168 and conduit 120. Thus, at early stages of cell culture process when the cell bag is relatively empty, the constrained volume may be closely coincident with the inner surface of the cell bag. But, as the liquid volume increases, the cell bag inflates, and the resultant wave motion of that liquid attains more energy during rocking motion of the cell bag, so then the constrained volume of the filter avoids the inner surface of the cell bag to prevent rubbing of the filter with the cell bag at that higher energy phase of the cell culture. Desirably, the tethers have a length such that there is always some clearance between the filter and the inner surface of the bag, for example at least 10 mm clearance. In practice this can be achieved by multiple tethers working in combination, where at least one will be taut while another is slack at the extremities of the filter's permitted range of movement. Additionally, in embodiments of a bioreactor of the present invention where conduit 120 extends between the filter and bottom layer 154, it is contemplated that conduit 120 will also have minimal contact with bottom layer 154 while also helping to support the filter in spaced separation from layer 154.

The predefined constrained volume 159 is thus generally depicted to represent an area within chamber 158 in which the filter 110 is constrained to remain within and where media fluid 151 is able to maintain the membranes 112 and 114 wetted desirably throughout operation of bioreactor 150, but at least while the waste contents are being evacuated from filter cavity 115 out conduit 120. Predefined constrained volume 159 is desirably defined to have separation from layers 152 and 154 while remaining below the surface of the rocking media 151. Tethers 168 desirably loosely maintains filter 110 under the surface of media 151 in that they do not fully constrain filter 110 against, e.g., layer 154 as filter clogging may in then arise. The present invention thus maintains the membranes of the filter fully wetted by the media to avoid bubble forming and filling of Waste bag with air which require customer to interrupt the process to add a new waste bag.

FIG. 8 depicts another bioreactor 350 of the present invention. Bioreactor 350 is contemplated to be similar to bioreactors 150 and 250, with like references indicating like components and with modifications as noted. Bioreactor 350 employs an alternate arrangement for the tethers 168 of the present invention. FIG. 8 depicts the application of multiple tethers 168 along the length of conduit 120 with first and second ends 170 and 172 both bonded to bottom layer 154 so as to define an open passageway 176 through which conduit 120 passes. In this embodiment, tethers 168 are generally aligned with the transverse central axis of bioreactor 350 so that conduit 120 may run out to bioreactor port 162 located below edge 155a such that conduit 120 may run clear of the rocking platform on which it rests. In this manner a portion 120a of conduit 120 proximate filter 110 extends freely into chamber 158 yet still maintains filter 110 within a predefined constrained volume of chamber 158 in accordance with the present invention.

The present invention contemplates that while portion 120a of conduit 120 is depicted as naturally flexing so as to turn towards the predefined constrained volume of chamber 158, conduit 120 may be formed with either an imparted bend about portion 120a or include one or more elbow segments arranged to turn the conduit body as shown and described. Such elbow segments may be bonded together by a suitable means of the prior art so as to be compatible with bioprocessing operations. Additionally, while port 118 and conduit 120 are shown to extend substantially normal to the planar filter 110, the present invention further contemplates that port 118 may provide for acute connection of conduit 120 to filter 110 so as to minimize the angle formed between conduit 120 and membrane 112. Such angled connection to conduit 120 may further reduce the minimum spacing between filter 110 and lower layer 154 of bioreactor 150.

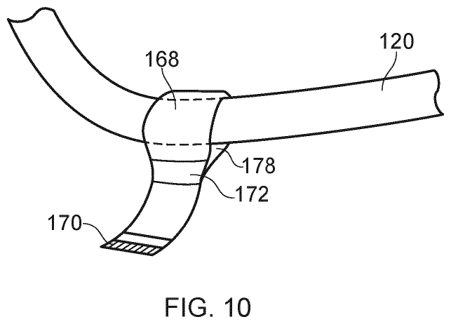

FIG. 9 depicts yet another bioreactor 450 of the present invention. Bioreactor 350 is contemplated to be similar to bioreactors 150, 250, and 350 with like references indicating like components and with modifications as noted. Bioreactor 450 arrangement for tethers 168 in which the second end 172 of each tether 168 may be suitably bonded to the outer surface of the conduit 120. While the tether 168 in FIG. 9 do not define an enclosed passageway for conduit 120, each still are desirably generally aligned along a transverse axis of bioreactor 450 so as to direct conduit 120 towards a bioreactor port 162 located below edge 155a so as to mitigate any effects a rocker platform supporting bioreactor 450 might have on flow through conduit 120. Portion 120a of conduit 120 will also desirably bend so as to allow the substantially planar body of filter 110 to extend generally parallel to the rocker on which bioreactor 450 sits. The present invention further contemplates that the shape of portion 120a of conduit 120, when membrane 112 is in facing opposition to lower layer 154, can further ensure separation of filter 110 from layer 154. Such shaping of portion 120a desirably ensures that filter 120 is fully wetted prior to evacuating waster therefrom through conduit 120 even in more shallow depths of media 151. With further reference to FIG. 10, the present invention contemplates that while the first end 170 of tether 168 is bonded to bottom layer 154, second end 172 may be looped about to be bonded to tether body 174 so as to define a passageway 178 through which conduit 120 passes. Each of the passageways 178 desirably deployed in bioreactor 450 are also generally aligned along a transverse axis of bioreactor 450 so as to direct conduit 120 towards bioreactor port 162 located below edge 155a so as to mitigate any effects a rocker platform supporting bioreactor 450 might have on flow through conduit 120. In the embodiments of both FIGS. 9 and 10, each tether 168 is sized and positioned so as to constrain a portion of conduit 120 such that the portion 120a of conduit 120 proximate, or connected to, filter 110 extends freely so as to confine the filter to a predefined constrained volume 159 of the bioreactor chamber in accordance with the present invention.

Desirably, the tethers and/or conduit are positioned and sized so as to maintain the filter in a submerged state when the bioreactor includes its operation fill of media, thus maintaining the filter membranes below the surface of the media and mitigating the risk of bubbling while also providing space to either above and below the portion of the conduit which is tethered so as to permit media flow past the conduit as the bioreactor bag is rocked. The present invention also contemplates that the positions and sizes of the tethers and conduit allow the filter to maintain the membranes as wetted so as to avoid bubbling while waste is evacuated from filter cavity 115 through conduit 120.

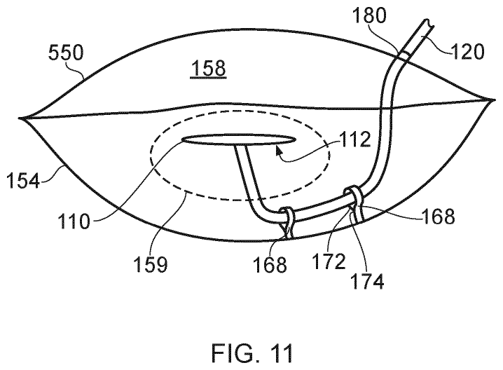

Moreover, the present invention further contemplates a bioreactor 550 as shown in FIG. 11. Bioreactor 550, desirably as properly tethered, provides that conduit 120 may extend from membrane 112 (i.e., the membrane in facing opposition to bottom surface 154) through a bioreactor port 180 located on the top surface 152 of bioreactor 150. Ends 172 of tethers 168 are looped onto their respective tether body 174, as was depicted in FIG. 10. Each of the passageways 178 desirably deployed in bioreactor 150 are also generally aligned along, as viewed from above, a transverse axis of bioreactor 150 so as to direct conduit 120 towards bioreactor port 180 located above edge 155a. Here again, filter 110 is loosely constrained to move within predefined constrained volume 159 of chamber 158.

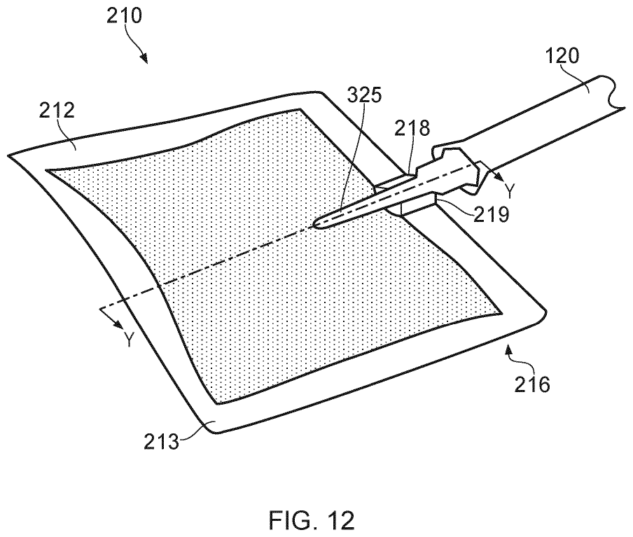

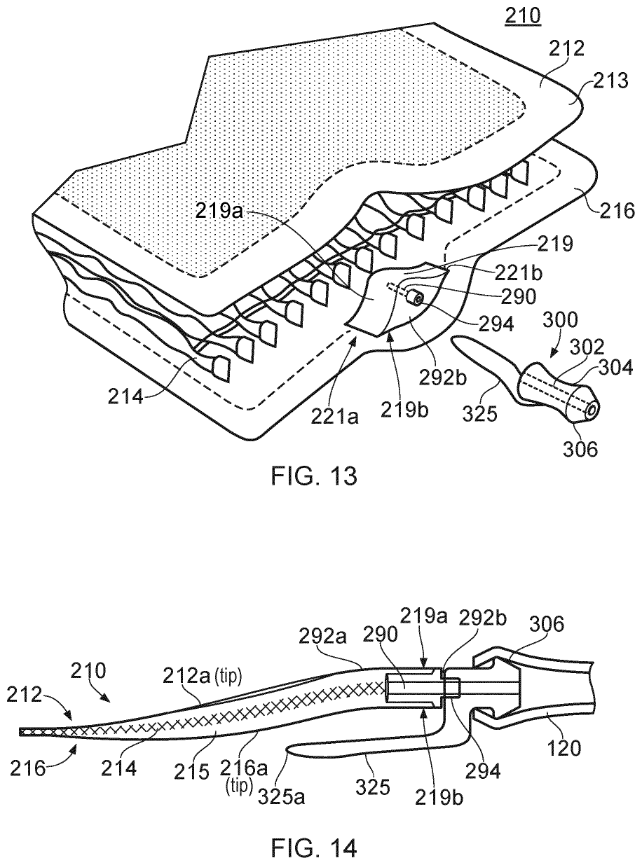

FIGS. 12-14 depict another substantially planar stacked filter 210 of the present invention. Filter 210, similar to filter 110, includes opposed planar membranes 212 and 216 perimetrically bonded at a sealed perimetrical edge 213 about an interior mesh 214. However, filter 210 provides a port 218 mounted between the perimetrical edges of membranes 212 and 216 at edge 213 so as to extend substantially co-planarly from filter 210, rather than substantially normal thereto as depicted for port 118 of filter 110.

The fitment body 219 of port 218 will thus be bonded to both membrane 212 and membrane 216. Fitment body 219 is known as a ‘boat fitment’ includes symmetrical, or opposed counter-tapering, surfaces 219a and 219b extending between opposed lateral edges 221a and 221b. Surfaces 219a and 219b are shaped to minimize the risk of any gaps forming between membranes 212 and 216 adjacent to edges 221a and 221b. Fitment body 219 defines an elongate open fitment passageway 290 extending therethrough in fluid communication so as to open on opposed fitment surfaces 292a and 292b. Passageway 290 further opens at the free end of a cylinder 294 protruding from surface 292b. When bonded together as shown, membranes 212 and 216 and port 218 thus define a filter cavity 215 within which is positioned a planar open mesh 214. Mesh 214 is shaped to allow for fluid flow from the pores 212a, 216a of membranes 212 and 216 through filter cavity 215 to and out of port 218.

Cylinder 294 is bonded to an adaptor body 300 which defines an elongate adaptor passageway 302 therethrough. Passageways 302 and 290 are thus placed in fluid communication with each other and thus with the pores 212a and 216a of membranes 212 and 216. The exterior surface 304 of adaptor body 300 is contoured to provide a tapered annular rim 306 over which one open end of conduit 210 is connected so as to conduct fluid from filter cavity 215 and out of the bioreactor in which it is positioned. Adaptor body 300 further supports a radially-displaced elongate projection 325 having a distal end 325a which is at least partially positioned in spaced registry with membrane 216. Desirably, adaptor body 300 is bonded to fitment body 219 after membranes 212 and 216 are bonded thereto, bonding mesh 214 within filter cavity 215, so that projection 325 does not interfere with properly bonding the membranes to the fitment body.

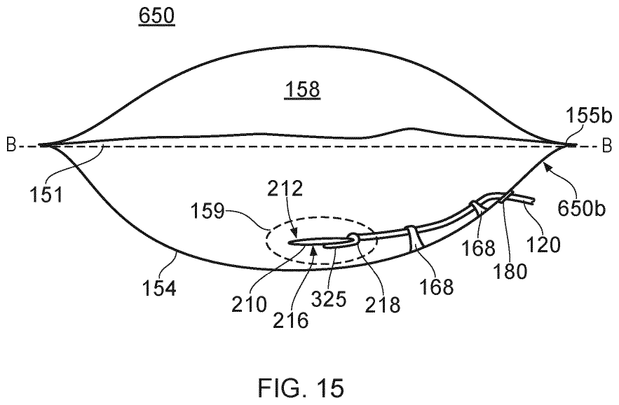

With additional reference to FIG. 15, filter 210 may be employed in a bioreactor 650 of the present invention. Bioreactor 650 is contemplated to be similar to bioreactors 150, 250, 350, 450 and 550 with like references indicating like components and with modifications as noted. Filter 210 is desirably bonded at port 218 to conduit 120 and optionally supported by one or more tethers 168 to a bioreactor layer 154 so as to eliminate spacing issues caused by portion 120a of conduit 120 as described hereinabove for filter 110, particularly for shallow depths of a media fluid 151. As conduit 120 extends substantially coplanar from membranes 212 and 216, the clearance between filter 210 and lower layer 154 of bioreactor 650 may be reduced. projection 325 is sized and shaped to ensure a minimum clearance between filter 210 and the lower layer 154 of bioreactor 650.

As shown in FIG. 15, conduit 120 may extend substantially along a longitudinal axis B-B of bioreactor 650 so as to exit bioreactor 650 at a location adjacent one longitudinal end, e.g., 650b, thereof. Conduit 120 extends through lower layer 154 through a bioreactor port 180 located below or otherwise adjacent edge 155b. The protrusion of conduit 120 through port 180 should be at an angle which permits flow through conduit 120 regardless of the angle of tilt applied to the bioreactor during operation. Moreover, other known filter designs may be used with the tethers of the present invention, such as that disclosed in commonly-assigned U.S. Provisional Patent Application Ser. No. 62/608,117, the entire contents of which are hereby incorporated by reference as if disclosed herein in its entirety. Such filters which eliminate the need for an elbow connector being connected between the filter and the conduit further reduce the risk of contact damage to the bioreactor.

Filter 210 may thus also be set to remain within predefined constrained volume 159 of bioreactor chamber 158 as has been described for the present invention. Mesh 214 of filter 210 is contemplated to be similar in design and construction as is suitable for mesh 114 of filter 110 and is desirably also formed to have some flexibility such that membranes 212 and 216 may deflect so as to remain submerged during rocking of bioreactor 650. Additionally, projection 325 protrudes a sufficient distance towards lower layer 154 to prevent the membranes of filter 210 to fully rest upon the lower layer 154 of bioreactor 150. Projection 325 desirably ensures that a minimum separation is maintained between the membranes of filter 210 and lower layer 154. The present invention further contemplates that projection 325 provides a rounded, blunt or non-sharp, shape so as to minimize risk of scratching or puncturing bioreactor 150 during transportation and storage, as well as during operation of bioreactor 150, should filter 210 touch upon lower layer 154 during rocking.

While the tethers of the present invention are each depicted as being used to properly position a stacked filter 110 of the present invention, it is also contemplated that the tethers of the present invention may be employed as shown and described with a single-membrane filter 10 of the prior art.

Additionally, the present invention further contemplates a combination of the rigidity of conduit 120 and the supporting arrangement for conduit 120 outside of a bioreactor of the present invention so as to maintain the filter within the predefined constrained volume of the present invention such that both membranes are wetted while waste is evacuated from filter cavity 115, 215.

In each embodiment of the present invention employing tethers, reference to a tether being bonded to a bottom layer 154 refers specifically to the tether being bonded to the surface of layer 154 facing bioreactor chamber 158. Additionally, for all embodiments, each bioreactor port 180 is bonded to both the bioreactor and to the conduit extending therethrough to prevent leaks and thus maintain fluid integrity of the bioreactor.

The invention is not to be seen as limited by the embodiments described herein, but can be varied within the scope of the appended claims as is readily apparent to the person skilled in the art. For example, in alternate embodiments, the filter could be attached to the inner surface of the bioreactor by two, three, four or more tethers. The filter could be of any suitable shape including but not limited to a square, a triangle or a circle. The filter could also have more than one port. The filter could be loosely tethered in various spatial orientations within the bioreactor chamber, with the aim that the filter can move within the bioreactor but not so much that the filter touches the inner surface of the bioreactor. Additionally, while the present invention has depicted conduit 120 as extending from filter 110 towards either upper layer 152 or lower layer 154 of the bioreactor, the present invention that in either case that conduit 120 may exit from either upper layer 152 or lower layer 154 of the bioreactor. Similarly, the conduit 120 extending from filter 210 is contemplated to exit the bioreactor through either upper layer 152 or lower layer 154 at a location suitable for the process to which the bioreactor is set to run, including but not limited to those locations described for bioreactors 150, 250, 350, 450, 550, or 650.

Moreover, while prior art mitigation techniques for mitigating the occurrence of bubbling may be employed with the present invention, the present invention further contemplates that the particular design and arrangement of the filter, conduit, and any tethers may be selected consistent with the present invention so as to mitigate bubbling apart from such prior art techniques.

While the particular embodiment of the present invention has been shown and described, it will be apparent to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

1. A bioreactor comprising:
a flexible bioreactor wall defining a bioreactor chamber, said wall further defining a bioreactor port therethrough;
an open bioreactor port fitment secured to said bioreactor wall at said bioreactor port;
a filter disposed within the bioreactor chamber, said filter comprising:
first and second overlying planar porous membranes perimetrically bound to each other so as to define a filter cavity therebetween,
a substantially planar open mesh disposed in said filter cavity,
wherein one of said first and second porous membranes defines an open aperture therethrough, and
an open outlet port fitment secured to said filter at said open aperture;
an elongate hollow conduit having a first portion affixed to said outlet port fitment of said filter and a second portion affixed to said bioreactor port such that said filter cavity is in fluid communication with said bioreactor port.

2. The bioreactor of claim 1, wherein said first porous membrane defines said open aperture.

3. The bioreactor of claim 1, wherein said bioreactor port fitment is secured to said first membrane so as to extend through said bioreactor port.

4. The bioreactor of claim 1, wherein said conduit sealingly extends through said port fitment on said outlet wall.

5. The bioreactor of claim 1, wherein said bioreactor port is located on an underside of said bioreactor.

6. The bioreactor of claim 5, wherein said bioreactor port is located about a transverse centerline of said bioreactor chamber.

7. The bioreactor of claim 6, wherein said bioreactor port is located below a seam formed by said bioreactor wall.

8. The bioreactor of claim 1, wherein said conduit assists in restricting movement of said filter to a predefined constrained volume of said bioreactor chamber.

9. The bioreactor of claim 1, wherein said bioreactor port is positioned below said filter, such that the length of said conduit will hold said filter submerged while said bioreactor chamber further includes a given amount of fluid.

10. The bioreactor of claim 1, wherein a portion of said conduit is affixed to a portion of said bioreactor wall below said filter whereby a free portion of the conduit extending from said bioreactor wall to said filter further operates as a tether for said filter to hold said filter submerged while said bioreactor chamber further includes a given amount of fluid.

11. The bioreactor of claim 1, further comprising at least one flexible tether having opposed ends affixed to said filter and said bioreactor wall below said filter thereby flexibly tethering the filter to the bioreactor chamber so as to allow the filter to move within a predefined constrained volume of the bioreactor chamber.

12. The bioreactor of claim 1, further comprising at least one elongate flexible tether having a first end affixed to said bioreactor wall and an opposed second end affixed to the body of the tether extending between said opposed ends, whereby said tether defines an open passageway through which said conduit passes.

13. The bioreactor of claim 1, further comprising at least one elongate flexible tether having opposed a first and second ends affixed to said bioreactor wall, whereby said tether and bioreactor wall define an open passageway through which said conduit passes.

14. The bioreactor of claim 8, wherein said constrained volume is spaced from an inner surface of the bioreactor chamber such that the filter does not touch an inner surface of the bioreactor chamber during operation of the bioreactor.

15. The bioreactor of claim 1, wherein said mesh provides a plurality of flow paths between each porous membrane and said conduit for conduct waste from said filter cavity.

16. The bioreactor of claim 1, further comprising a pump for selective engagement with said conduit so as to draw waste products from said bioreactor chamber through said filter cavity into said conduit.

17. A method of operating a bioreactor comprising:
providing a bioreactor of claim 1;
at least partially filling the bioreactor chamber with liquid media; and
rocking the bioreactor thereby inducing the filter to move relative to the bioreactor chamber such that the filter moves within the bioreactor chamber within a predefined constrained volume.

18. The method of claim 17, wherein said constrained volume is spaced from an inner surface of the bioreactor chamber such that the filter cannot touch an inner surface of the bioreactor chamber in use, while keeping the filter afloat or at least partly submerged in liquid media.

19. The method of claim 17, further comprising the step of pumping waste products from said bioreactor chamber through said filter into said filter cavity and out of said bioreactor through said conduit.

20. A bioreactor comprising:
a flexible bioreactor wall defining a bioreactor chamber, said wall further defining a bioreactor port therethrough;
an open bioreactor port fitment secured to said bioreactor wall at said bioreactor port;
a filter disposed within the bioreactor chamber, said filter comprising:
at least one porous membrane at least partially bounding a filter cavity, and
an open outlet port fitment secured to said filter;
an elongate hollow conduit having a first portion affixed to said outlet port fitment of said filter and a second portion affixed to said bioreactor port such that said filter cavity is in fluid communication with said bioreactor port;
at least one elongate flexible tether having a first end affixed to said filter and said bioreactor wall, said tether flexibly tethering the conduit along the bioreactor wall so as to allow the filter to move within a predefined constrained volume of the bioreactor chamber.

21. The bioreactor of claim 20, wherein said at least one elongate flexible tether includes a first end affixed to said filter and an opposed second end affixed to said bioreactor wall below said filter.

22. The bioreactor of claim 20, wherein said at least one elongate flexible tether includes a first end affixed to said bioreactor wall and an opposed second end affixed to the body of the tether extending between said opposed ends, whereby said tether defines an open passageway through which said conduit passes.

23. The bioreactor of claim 20, wherein said at least one elongate flexible tether includes opposed a first and second ends affixed to said bioreactor wall, whereby said tether and bioreactor wall define an open passageway through which said conduit passes.

24. A filter for use within a bioreactor chamber, said filter comprising:
first and second overlying planar porous membranes perimetrically bound to each other so as to define a filter cavity therebetween,
a substantially planar open mesh disposed in said filter cavity,
wherein one of said first and second porous membrane defines an open aperture therethrough, and
an open outlet port fitment secured to said filter at said open aperture.

25. A filter for use within a bioreactor chamber, said filter comprising:
first and second overlying planar porous membranes each including a first portion perimetrically bound to each other,
a substantially planar open mesh disposed in said filter cavity,
wherein each of said first and second porous membranes include a second portion affixed to a fitment body positioned therebetween, wherein said first and second membranes and said fitment body define a filter cavity therebetween defines an open aperture therethrough, and
an open outlet port defined by said fitment body, said outlet port extending in fluid communication with said filter cavity.

26. The filter of claim 25, wherein said fitment body further includes a blunt projection depending therefrom so as to ensure a fixed stand-off of said filter from a wall defining the bioreactor chamber.
